(19) United States
(12) Patent Application Publication
(10) Pub. No.: US 2005/0238643 A1
(43) Pub. Date: Oct. 27, 2005

(54) Modulation of lir function to treat rheumatoid arthritis

(76) Inventors: Jonathan P. Arm, Boston, MA (US); Nicodemus Tedla, Sydney (AU); Luis Ghira Borges, Oak Park, CA (US)

Correspondence Address: IMMUNEX CORPORATION, PATENT OPERATIONS/MS 28-2-C, ONE AMGEN CENTER DRIVE, THOUSAND OAKS, CA, 91320-1799, US

(21) Appl. No.: 10/495,695

(22) PCT Filed: Nov. 12, 2002
(86) PCT No.: PCT/US02/36372

§ 371 Date: Jun. 24, 2005

Related U.S. Application Data
(60) Provisional application No. 60/335,899, filed on Nov. 14, 2001.

(51) Int. Cl. A61K 39/395

(52) U.S. Cl. 424/143.1

(57) ABSTRACT

This invention relates to methods for treating rheumatoid arthritis (RA) by administering one or more agents that specifically target LIR-2, LIR-3 and LIR-7. Results disclosed herein indicate that these three LIRs may participate in regulating the activation of leukocytes that infiltrate synovial tissue. Thus, inflammation in the joints of RA patients can be ameliorated by administering agents that modulate expression or function of LIR-2 and/or LIR-3 and/or LIR-7 to reduce or eliminate the activation of monocytes or macrophages present in inflamed joints, or to reduce their recruitment to the site of inflammation. This can be accomplished by modulating LIR-7, which transmits a stimulatory signal, and/or by modulating LIR-2 and/or LIR-3, which when triggered exert an inhibitory effect, or by concurrently modulating two or all three of these LIRs.

### BACKGROUND OF THE INVENTION

Rheumatoid arthritis (RA) is a chronic inflammatory synovitis that is characterized by synovial hypertrophy and synovial pannus formation with accompanying destruction of juxtaarticular cartilage and bone (Tak, P P, Arthritis & Rheumatism 43(12):2619-33 (2000)). The predominant inflammatory cells found at inflamed sites are macrophages (type A synoviocytes) and fibroblast-like cells (type B synoviocytes), as well as increased numbers of neutrophils, mast cells, natural killer cells, plasma cells, and lymphocytes (Leirisalo-Repo et al., Inflammation 17(4):427-42 (1993); Gotis-Graham I and McNeil HP, Arthritis & Rheumatism 40(3):479-89 (1997); Malone et al., Arthritis & Rheumatism 30(2):130-37 (1987); Bromley M and Wooley D E, Arthritis & Rheumatism 27:857-63 (1984); Jefferis R., British Medical Bulletin 51(2):312-31 (1995)). These cells apparently promote inflammation and tissue destruction by releasing multiple factors such as lipid mediators (Elmgreen et al., Ann Rheum Dis 46:501-05 (1987); Moilanen E., Pharmacol Toxicol 75 (Suppl 2):4-8 (1994)), pro-inflammatory cytokines (Firestein et al., J Immunol 144:3347-53 (1990); Westacott et al., Ann Rheum Dis 49:(9)676-81 (1990); Alvaro-Gracia et al., J Clin Invest 86:1790-98 (1990); Brennan et al., Br J Rheumatol 30 (Suppl 1):76-80 (1991)) and tissue degrading enzymes (Hembry et al., Ann Rheum Dis 54(1):25-32 (1995); Taylor et al., Ann Rheum Dis 53(1):768-72 (1994)).

Synovial macrophages are the predominant source of interleukin IL-1β and tumor necrosis factor TNFα, which are central to the pathogenesis of RA as evidenced by the efficacy of disease modifying therapies targeted at these cytokines (Feldmann M and Maini R N, Ann Rev Immunol 19:163-96 (2001)). The joint destruction in RA is likely mediated by proteases derived from macrophages and osteoclasts. Although there is abundant evidence for the presence of activated leukocytes in rheumatoid synovium, the mechanism(s) and regulation of their activation are not well understood.

A new family of proteins termed “leukocyte immunoglobulin-like receptors” (LIRs), or “immunoglobulin-like transcripts (ILTs),” are expressed on the surfaces of various cells involved in inflammation and immune responses. Members of this family have been shown in vitro to modulate cellular responses through immunoreceptor tyrosine-based inhibitory motifs (ITIMs) present in their cytoplasmic tails, or through association in the cell membrane with other proteins that contain an “immunoreceptor tyrosine-based activation motif,” or “ITAM motif” (Arm et al., J Immunol 159:2342 (1997); Fanger et al., Eur J Immunol 28:3423-34 (1998); Borges et al., J Immunol 159:5192-96 (1997); Samaridis J and Colonna M, Eur J Immunol 27:660-65 (1997); WO 98/48017; U.S. Pat. No. 6,140,076; and WO 00/68383).

The range of cellular responses regulated by LIRs has been studied in vitro. It has been shown that the recognition of MHC class-I molecules by LIR-1 or LIR-2 inhibits NK cell activity and T cell cytotoxicity (see, for example, Colonna et al., J Exp MedI 186:1809-18 (1997); Fanger et al., 1998). Co-ligation of inhibitory LIR-1, LIR-2, LIR-3 or LIR-5 with an activating receptor such as the BCR, TCR, FcγR or MHC class I molecules led to inhibition of Ca²⁺ flux and subsequent down-stream events elicited by the activating molecule (Cella et al., J Exp Med 185:1743-51; Colonna et al., 1997; Colonna et al., 1998; and Saverino et al., J Immunol 165:3742-55 (2000)). These events have recently been elucidated for the interaction of LIR-1 with the T cell receptor (Dietrich et al., J Immunol 166:2514-21 (2001)).

It has been suggested that LIRs and related molecules may determine the threshold and/or extent of activation of leukocytes in inflammatory conditions. This idea is supported by recent studies in mice in with disruption of gp49B 1, which is a protein with similarities to the LIRs (Daheshia et al., J Exp Med 189:309-318 (2001)).

The inhibitory LIRs, which include LIRs-1, 2, 3, 5 and 8, display long cytoplasmic domains that contain two to four ITIMs. LIR-2, also known as “ILT4” or “MIR10,” contains three ITIM motifs and binds a diverse array of MHC class I proteins, including HLA-A, HLA-B and HLA-C alleles (Fanger et al., 1998). LIR-1 and LIR-2 are known to interact with class I molecules with broad specificity, recognizing alleles within HLA-A, B, C and HLA-G (Colonna et al., 1998; Cosman et al., Immunity 7:273-82 (1997); Banham et al., J Leukocyte Biol 65:841-45 (1999)). One means by which the inhibitory LIRs mediate inhibition of cell activation is by recruiting the src homology 2 (SH2) domain-containing phosphatase 1 (SHP-1) to inhibit or terminate signaling through non-receptor tyrosine kinase cascades (for example, see Cella et al., 1997; Colonna et al., 1997; Colonna et al., J Immunol 160:3096-3100 (1998); and Dietrich et al., J Immunol 166:2514-21 (2001)).

The activating LIRs, which include LIR-6a, LIR6b and LIR-7, are characterized by a short cytoplasmic domain and a positively charged arginine residue within the transmembrane domain that may facilitate association with FcεRIγ. LIR-7 is also called “ILT1.” Although LIR-7 itself contains a rather short cytoplasmic tail, it specifically associates in the cell membrane with the Fc receptor gamma chain (FcεRIγ) (Nakajima et al., J Immunol 162:5-8 (1999)). This Fc receptor gamma chain has a cytoplasmic tail that contains an ITAM, thus can transmit a stimulatory signal. FcεRIγ is known to serve as a signaling partner for other proteins, namely, FCαR and activating NK cell receptors. Nakajima et al. propose that LIR-7 activates cells using the associated protein FcεRIγ to transduce the stimulatory signal. The positively charged arginine residue within the transmembrane domain of LIR-7 that may facilitate association with FcεRIγ. Nakajima et al. demonstrated the capacity of LIR-7 to mediate cell activation by showing that it could trigger serotonin release in rat basophilic leukemia cells (RBL cells) that were expressing transfected LIR-7. The serotonin release was triggered by exposing the cells to antibodies that cross-linked the cell-surface LIR-7. In addition, this group demonstrated that cross-linking LIR-7 elicited intracellular calcium mobilization in several cell types, including primary monocytes, P815 cells transformed with a LIR-7-expression vector, and the transfected RBL cells (Nakajima et al., 1999). Mobilization of calcium is an early event in monocyte activation.

The cellular distribution of LIR-1, LIR-2, LIR-5 and LIR-7 has been studied in detail using monoclonal antibodies. LIR-1 is expressed on all peripheral blood monocytes, in vitro-derived dendritic cells and macrophages, B cells, and a subset of T cells and NK cells (Samaridis et al., 1997; Cosman et al., 1997). A more restricted cellular distribution was reported for LIR-2 and LIR-5, which are most prominent on monocytes and dendritic cells (Colonna et al., 1998). LIR-7 is expressed in all peripheral blood monocytes and granulocytes, in vitro-derived macrophages and in dendritic cells (Nakajima et al., 1999). At the mRNA level, transcripts for LIR-3 and LIR-6 were detected in monocytes and B cells, transcripts for LIR-4 were detected in B cells, NK cells and monocytes, while transcripts for LIR-8 were detected only in NK cells (Borges et al., 1997; Arm et al., 1998). Thus, the therapeutic modulation of LIR expression is a promising area for exploration.

### SUMMARY OF THE INVENTION

Provided herein are methods for treating RA by administering one or more agents that specifically target LIR-2, LIR-3 and LIR-7. Results disclosed herein indicate that these three LIRs may participate in regulating the activation of leukocytes that infiltrate synovial tissue. Thus, inflammation in the joints of RA patients can be ameliorated by administering agents that modulate expression or function of LIR-2 and/or LIR-3 and/or LIR-7 to reduce or eliminate the activation of monocytes or macrophages present in inflamed joints, or to reduce their recruitment to the site of inflammation. This can be accomplished by modulating LIR-7, which transmits a stimulatory signal, and/or by modulating LIR-2 and/or LIR-3, which when triggered exert an inhibitory effect, or by concurrently modulating two or all three of these LIRs.

Patients having RA are treated in accord with the invention by administering an effective amount of an agonistic agent that binds LIR-2 or LIR-3, or by concurrent administration of agonists that bind each of these proteins thereby triggering their inhibitory activity. Administration of a LIR-2 agonist and/or a LIR-3 agonist to a RA patient will result in a diminution in calcium mobilization in synovial monocytes in the patient. In a preferred embodiment of the invention, the agent is an agonistic antibody that is specifically immunoreactive with LIR-2. In another preferred embodiment, the agent is an agonistic antibody that is specifically immunoreactive with LIR-3. Monoclonal antibodies are preferred. Preferably, the monoclonal antibody is a humanized antibody in which the variable region is derived from a rodent and the remainder from a human. More preferably, the monoclonal antibodies are fully human. In other embodiments, the LIR-2 or LIR-3 agonist is a small organic molecule.

In another aspect of the invention, RA patients are treated with an agent that antagonizes the biological activity of LIR-7. Such an agent will partially or completely block LIR-7 from becoming triggered, thus preventing it from activating monocytes or macrophages at the site of an inflamed joint. Administration of a LIR-7 antagonist to a RA patient will result in a diminution in the amount of calcium released from synovial monocytes in the patient. In one aspect of the invention, the agent is an antagonistic antibody that is specifically immunoreactive with LIR-7, that is, the antibody binds specifically with an epitope that is unique to LIR-7. Such antibodies are blocking antibodies, and block the binding of LIR-7 to its ligand. Preferably, the antibody is a monoclonal antibody. In a preferred embodiment, the monoclonal antibody is a humanized antibody, and it another preferred embodiment, the monoclonal antibody is fully human. In another embodiment, the LIR-7 antagonist is a soluble polypeptide comprising the extracellular region of LIR-7, including fusion proteins in which this extracellular region is joined with the Fc region of a human immunoglobulin protein. In yet other embodiments, the LIR-7 antagonist is a small organic molecule. The LIR-7 antagonist may be administered alone or together with an agonist of LIR-2 and/or LIR-3.

Other aspects of the invention provide methods of treatment that involve concurrent treatment of RA with various combinations of agonistic agents that trigger LIR-2 and/or LIR-3 administered concurrently with each other or administered concurrently with agents that antagonize LIR-7. This includes treatments that comprise the concurrent administration of one of the following combinations: i) an agent that agonizes LIR-3 and an agent that antagonizes LIR-7; ii) an agent that agonizes LIR-2 and an agent that antagonizes LIR-7; iii) an agent that agonizes LIR-2 and an agent that agonizes LIR-3; iv) an agent that agonizes LIR-2, an agent that agonizes LIR-3 and an agent that antagonizes LIR-7. Preferably, such agents are monoclonal antibodies, including humanized or human antibodies. Other preferred agents are small organic molecules that agonize LIR-2 and/or LIR-3, or that antagonize LIR-7. In addition, methods of treatment are provided that combine the aforedescribed LIR modulators with the concurrent use of other medications used to treat rheumatoid arthritis.

### DETAILED DESCRIPTION

It is shown herein using immunohistochemical techniques that LIR-2, LIR-3 and LIR-7 are differentially expressed in leukocytes infiltrating the synovium of patients with RA. RA patients are people who have one or more inflamed or tender joints and whose serum tests positive for rheumatoid factor. Inflammatory responses, such as those seen in RA patients, are likely regulated by a complex network of inhibitory and activating signals. Thus, provided here are therapeutic methods comprising administration of agents that modulate the expression in RA synovium of LIR-2, LIR-3 or LIR-7. In one embodiment of the invention, the disclosed therapeutic treatments are administered to RA patients whose synovial tissues express elevated levels of LIR-2, LIR-3 or LIR-7 as compared with non-RA patients (control tissues). To determine whether levels of these proteins are elevated, tissue from synovial biopsies is analyzed by staining with antibodies against these LIRs. Agents that inhibit or enhance a biological activity of LIR-2, LIR-3 or LIR-7 are referred to herein as “LIR modulators.”

Observations disclosed herein indicate that LIR-2, LIR-3 and LIR-7 are expressed at elevated levels in leukocytes infiltrating the synovium of patients with RA, but are not elevated in synovium of patients who have osteoarthritis (OA). RA is characterized by extensive infiltration into synovial tissues of leukocytes that mediate inflammation, while OA is a chronic degenerative condition not usually associated with inflammation. The disclosed observations indicate also that expression of LIR-2, LIR-3 and LIR-7 is lower in RA patients with established fibrosis than in RA patients in earlier stages of the disease. Generally, fibrosis develops only in RA patients who have been afflicted for a number of years. For example, those afflicted for eight or more years are likely to have developed fibrotic joints. In one embodiment of the invention, LIR modulators are administered to RA patients whose joints have not become fibrotic.

As used herein, the term LIR-2 encompasses a polypeptide having an amino acid sequence as shown in SEQ ID NO:2, as well as variants and muteins having the biological properties of this protein. The LIR-2 shown in SEQ ID NO:2 has a predicted extracellular region of 458 amino acids (amino acids 1-458 of SEQ ID NO:2) including a 16 amino acid signal peptide at amino acids 1-16. This LIR-2 also includes a transmembrane domain at amino acids 459-483 of SEQ ID NO:2, and a cytoplasmic domain that includes amino acids 484-598 of SEQ ID NO:2. The extracellular domain includes four immunoglobulin-like domains and the cytoplasmic domain includes three ITIM motifs at amino acids 531-536, 560-565 and 590-595.

The term “LIR-2,” as used herein, also includes a polypeptide having an at least 90% amino acid sequence identity, or more preferably an at least 95%, or most preferably an at least 98% amino acid sequence identity with amino acids 17-458 of the LIR-2 shown in SEQ ID NO:2, and also having a biological activity associated with a LIR having the amino acid sequence shown in SEQ ID NO:2. One example of such a biological activity is the ability to bind to a MHC class I polypeptide. The ability of an LIR-2 polypeptides to bind MHC I may be determined by any convenient assay, such as an assay that detects binding of the LIR-2 polypeptide to a MHC class I protein that is expressed on the surface of cells. The specificity of the binding can be ascertained by testing whether antibodies specific for MHC I can block a putative LIR-2 from binding to cells that are known to express MHC I. Cells and cell lines that may be used for such tests include any cells expressing a MHC I, such as CB23, HSB2, MP-1, Jurkat, primary T cells, primary B cells or primary NK cells.

A suitable assay for detecting LIR-2 binding to MHC I is the flow cytometry assay described in WO 98/48017, which is hereby incorporated by reference in its entirety. In brief, to perform this assay, cells expressing MHC I are first washed with FACS buffer (2% FCS in PBS with 0.2% azide), then aliquots of about 10⁵ cells are incubated for about one hour in 100 μL blocking buffer (2% FCS, 5% NGS, 5% rabbit serum in PBS). To each cell sample is added increasing amounts (e.g., 0, 2, 5, or 10 μg) of control serum or W6/32 (ATCC HB-95) in 100 μL blocking buffer. W6/32 is an antibody specific for MHC class I heavy chains, including HLA-A, B, and C polypeptides, and preincubation with this antibody will competitively block the specific binding of other proteins to MHC I. Following the addition of the W6/32 or control serum, the samples are incubated on ice for about one hour and then washed about three times with 200 μL of FACS buffer. Next, about 5 μg of a LIR-2 polypeptide is added. The LIR-2 polypeptide for this assay is a soluble fusion protein comprising the extracellular region of the LIR-2 joined with the Fc region of a human IgG immunoglobulin protein (sLIR-2/Fc). The sLIR-2/Fc polypeptide in blocking buffer is added to each sample and the mixture incubated on ice for about one hour. Following incubation with the sLIR-2/Fc, the cells are washed several times with FACS buffer and treated for about 45 minutes with biotin-tagged mouse antibody that is specifically immunoreactive with the Fc region of human IgG (available from Jackson Research Laboratories) and SAPE (streptavidin-phycoerythrin; available from Molecular Probes). SAPE is a fluorescing compound that will bind to the biotin moiety of the anti-human Fc/biotin, and that will fluoresce when exposed to the appropriate excitation and emission conditions. Thus, the biotinylated anti-human Fc reacts with the cell-bound sLIR-2/Fc, and the SAPE in turn binds to the biotin moiety of the anti-human Fc. To detect cells to which the sLIR-2/Fc has bound, cells exposed to the LIR-2/Fc, the anti-human Fc and SAPE are washed with FACS buffer and subjected to flow cytometry to detect SAPE-tagged cells. If the sLIR-2/Fc binds to the cells but is competitively prevented doing so by W6/32, this indicates that the sLIR-2/Fc is capable of specific binding to MHC I.

Percent amino acid sequence identity as used herein is determined by dividing the number of aligned amino acids that are identical by the total number of amino acids in the shorter of the two sequences being compared. A number of computer programs are available commercially for aligning sequences and determining sequence identities and variations. These programs provide identity information based upon the above stated definition of identity. One suitable computer program is the GAP program, version 6.0, described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for amino acids, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pages 353-358, 1979; (2) a penalty of 3.0 for each gap and an additional 0.10 penalty for each symbol in each gap; and (3) no penalty for end gaps. Also useful for these comparisons is another similar program called BESTFIT, which also is available from the University of Wisconsin as part of the GCG computer package for sequence manipulation.

The term “LIR-2,” as used herein, further refers to a polypeptide that is encoded by a nucleic acid molecule having the nucleotide sequence shown in SEQ ID NO:1. Such nucleic acid molecules include single stranded and double-stranded DNA. Also included are RNA molecules having the equivalent sequence but with “U” being substituted for “T.” LIR-2 polypeptides encoded by such nucleic acids are capable of binding MHC I.

Also encompassed by the term “LIR-2” are polypeptides that possess a biological activity exhibited by the LIR-2 shown in SEQ ID NO:2 and that are encoded by nucleic acid molecules whose complements are capable of hybridizing under moderately stringent or under highly stringent conditions with a nucleic acid molecule having the nucleotide sequence shown in SEQ ID NO:1. Highly stringent hybridization conditions are designed to minimize the formation of mismatched base pairs. The basic parameters affecting the stringency of hybridization conditions and guidance for devising suitable conditions are set forth by Sambrook, J., E. F. Fritsch, and T. Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, F. M. Ausubel et al., eds., John Wiley & Sons, Inc., sections 2.10 and 6.3-6.4). Hybridization can be performed in solution or by anchoring the target nucleic acid to a solid surface, such as a nitrocellulose or nylon filter. The DNA to be tested is labelled, denatured and used as a probe in the hybridization reaction. Conditions of any desired degree of stringency can be readily determined by those having ordinary skill in the art based on, for example, the length and/or base composition of the DNA. One way of achieving moderately stringent conditions for filter-bound target DNA involves using a prewashing solution containing 5×SSC, 0.5% SDS, 1.0 mM EDTA (pH 8.0), hybridization buffer containing 6×SSC, and a hybridization temperature of about 68° C. (or using a hybridization buffer containing 50% formamide and 6×SSC, and hybridization temperature of about 42° C.), then washing the filters at about 60° C. in a buffer containing 0.5×SSC and 0.1% SDS. “SSC” (1×) is 0.15 M NaCl, 0.015 M Na citrate, pH 7.0. Generally, highly stringent conditions are defined as above, except that the wash step is conducted in 0.2×SSC/0.1% SDS at about 68° C. If desired, SSPE (1×SSPE is 0.15M NaCl, 10 mM NaH₂PO₄, and 1.25 mM EDTA, pH 7.4) can be substituted for SSC in the above-described hybridization and wash buffers, and the SDS can be increased or omitted from any of the hybridization or wash buffers without affecting the stringency. Biological activities possessed by LIR-2 proteins encoded by these nucleic acids include one of more of the following: the ability to bind MHC class I proteins; the ability when triggered to reduce intracellular calcium flux in activated monocytes; and the ability when triggered to reduce the level of intracellular phosphorylated tyrosine in activated monocytes.

In addition, LIR-2 polypeptides according to the invention include proteins that are capable of binding MHC I and that are immunoreactive with an antibody that is specifically immunoreactive with a polypeptide having the amino acid sequence shown in SEQ ID NO:2. An antibody that is specifically immunoreactive with LIR-2 is one that shows no appreciable binding with other proteins. For example, antibodies specific for LIR-2 will not bind with LIR-3 or LIR-7. Methods for producing specific antibodies are well-known in the art, and any suitable method may be used to produce such antibodies, such as the methods discussed below. Generally, LIR-2 polypeptides that react with such antibodies are encoded by a nucleic acid capable of hybridizing under stringent conditions with a nucleic acid having the nucleotide sequence shown in SEQ ID NO:1.

As used herein, the term “LIR-3” encompasses a polypeptide having the amino acid sequence shown in SEQ ID NO:4, as well as variants and muteins having the biological properties of this protein. The LIR-3 polypeptide of SEQ ID NO:4 can be encoded, for example, by a nucleic acid molecule having the nucleotide sequence shown in SEQ ID NO:3. The term “LIR-3” also includes variants having an at least 90%, or more preferably an at least 95%, or most preferably an at least 98% amino acid sequence identity with SEQ ID NO:4, and further possessing a biological activity expressed by a polypeptide with the amino acid sequence shown in SEQ ID NO:4. Such biological activities include the ability to reduce intracellular calcium flux in activated monocytes and the ability to reduce the amount of phosphorylated tyrosine in proteins present in activated monocytes. Percent identity is determined as described above. The LIR-3 amino acid sequence presented in SEQ ID NO:4 has an extracellular domain containing amino acids 1-443, which includes a signal peptide of amino acids 1-16; a transmembrane domain that includes amino acids 444-464; and a cytoplasmic domain having amino acids 465-631. The extracellular region of the LIR-3 polypeptide of SEQ ID NO:4 contains four immunoglobulin-like (Ig-like) domains, and its cytoplasmic region contains two pairs of ITIM motifs. In SEQ ID NO:4, a first pair of ITIM motifs is located at amino acids 512-517, 541-546, and a second pair is at amino acids 593-598 and 623-628. As used herein, the term “LIR-3” thus encompasses a polypeptide having four Ig-like domains in its extracellular region, four ITIM motifs in its cytoplasmic tail, and having at least one biological activity exhibited by an LIR-3 having the amino acid sequence shown in SEQ ID NO:4. LIR-3 polypeptides according to the invention are capable of transmitting an inhibitory stimulus that suppresses calcium release in monocytes expressing this protein.

LIR-2 and LIR-3 are inhibitory proteins. Biological activities of these proteins include the capacity to inhibit monocytes that have been activated. In one method for assaying for this activity of LIR-2 or LIR-3, monocytes are first activated by being exposed to anti-CD64 antibody. This activation is accompanied by an intracellular release of calcium and by the phosphorylation of tyrosine residues on proteins in these cells. LIR-2 and LIR-3 are expressed on the surface of the activated monocytes. Triggering LIR-2 or LIR-3 on these monocytes can be accomplished, for example, by exposing the cells to an agonistic antibody specific for either LIR-2 or LIR-3. In some instances, triggering will also involve adding a second antibody that reacts with the anti-LIR antibody, resulting in cross-linking the LIR that is expressed on the cell surface. This cross-linking will trigger the inhibitory function of the LIR-2 or LIR-3, thus resulting in reduced calcium flux, which can be assayed as described below.

The term “LIR-7” encompasses the polypeptide whose amino acid sequence is shown in SEQ ID NO:6, as well as variants and muteins having the biological properties of this protein. The amino acid sequence presented in SEQ ID NO:6 has an extracellular domain that includes amino acids 1449, a signal peptide from amino acids 1-16, a transmembrane domain that includes amino acids 450468 with a charged arginine residue at amino acid 452 and a short cytoplasmic domain at amino acids 469-483. The extracellular domain includes four immunoglobulin-like domains.

In one aspect of the invention, the LIR-7 polypeptide is encoded by a nucleic acid molecule having the nucleotide sequence shown in SEQ ID NO:5. In addition, the term “LIR-7” encompasses polypeptides encoded by nucleic acids capable of hybridizing under moderately stringent or highly stringent conditions with the complement of a nucleic acid having the nucleotide sequence shown in NO:5, and further having a biological activity possessed by a protein having the amino acid sequence shown in SEQ ID NO:6. Hybridization conditions are defined as described above.

LIR-7 polypeptides according to the invention possess stimulatory capacity (see, for example, Nakajima et al., 1999). Triggering a LIR-7 polypeptide stimulates serotonin release from rat basophilic leukemia cells, and stimulates calcium release from primary monocytes or other cells expressing this protein. Calcium release is a measurable indicator of monocyte activation, and can be detected, for example, using the assay in Nakajima et al. for monitoring calcium cytoplasmic levels in individual cells (1999). To perform this assay, cells are loaded with Indo-1 AM dye (Sigma, St. Louis, Mo.) and analyzed on a flow cytofluorometer as previously described (see Colonna et al., 1997 and Nakajita et al., 1999). In brief, a baseline is acquired prior to pausing the analysis for the addition of antibodies that cross-link LIR-7. Cross-linking generally requires adding a monoclonal antibody against LIR-7 and an antibody directed against the IgG from the animal species in which the anti-LIR-7 antibody was raised. Analysis consists of measuring 405/525 spectral emission ratio of the loaded Indo-1 dye by flow cytometry.

Serotonin release is another indicator that can be used to measure LIR-7 activity. Serotonin release is assayed as previously described in Colonna et al., 1997. In brief, the cells are pulsed with H³-serotonin (5-hydroxytryptamine), washed, then contacted with antibodies against serotonin and either antibodies that cross-link LIR-7 on the cell surface or with a control antibody to correct for spontaneous release. Serotonin release is calculated according to the formula: % serotonin release=100×([cpm sample]−[cpm spontaneous release])/(cpm total).

The term “LIR-2” as used herein also encompasses polypeptides that are immunoreactive with antibodies that bind specifically with a polypeptide having an amino acid sequence according to SEQ ID NO:2 and that further possess a biological activity characteristic of a protein with the amino acid sequence shown in SEQ ID NO:2. The term “LIR-3” as used herein also encompasses polypeptides that are immunoreactive with antibodies that bind specifically with a polypeptide having an amino acid sequence according to SEQ ID NO:4 and that further possess a biological activity characteristic of a protein with the amino acid sequence shown in SEQ ID NO:4. The term “LIR-7” encompasses polypeptides that are immunoreactive with antibodies that bind specifically with a polypeptide having an amino acid sequence according to SEQ ID NO:6 and that further possess a biological activity characteristic of a protein with the amino acid sequence shown in SEQ ID NO:6.

Therapeutic Agents

Therapeutic agents useful for the disclosed therapeutic methods include non-toxic agents capable of increasing the level of expression or function of LIR-2 or LIR-3, and non-toxic agents capable of decreasing the level of expression or function of LIR-7. Exemplary agents for this purpose include agonistic antibodies specifically immunoreactive with LIR-2 or LIR-3 and antagonistic antibodies specifically immunoreactive with LIR-7. In other embodiments, the therapeutic agent is a soluble polypeptide comprising part or all of the extracellular region of LIR-7, including fusion proteins in which the extracellular region of LIR-7 is joined with the Fc region of a human IgG immunoglobulin (sLIR-7:Fc). The Slir-7:Fc will act as a competitive inhibitor of native LIR-7. In one aspect of the invention, the therapeutic agent is a polypeptide in which amino acids 1-449 or 17-449 of SEQ ID NO:6 is fused with an IgG1 Fc region as described above.

To raise antibodies specific for one of the LIRs, one may use as an antigenic stimulus the entire LIR, or a polypeptide comprising the extracellular region of LIR-2, LIR-3 or LIR-7 or an antigenic subportion thereof. The extracellular regions of LIR-2, LIR-3 and LIR-7 correspond, respectively, to amino acids 1-458 of SEQ ID NO:2, amino acids 1-443 of SEQ ID NO:4 and amino acids 1-449 of SEQ ID NO:6. Amino acids 1-16 of SEQ ID NOS:2, 4 and 6 correspond to signal sequences that are cleaved during maturation of the LIR. Full-length LIR-2, LIR-3 or LIR-7 or their extracellular regions with or without the signal sequence may be used for raising antibodies. Extracellular regions of LIR-2, LIR-3 and LIR-7 lacking a signal peptide would correspond, respectively, to amino acids 17458 of SEQ ID NO:2, 17-443 of SEQ ID NO:4 or 17-449 of SEQ ID NO:6. Additionally, antibodies can be raised using subportions of LIR-2, LIR-3 or LIR-7 that contain at least one antigenic epitope that is unique to that LIR.

Polyclonal and monoclonal antibodies for use as therapeutic agents may be prepared by conventional techniques (see, for example, Monoclonal Antibodies, Hybridomas: A New Dimension in Biological Analyses, Kennet et al. (eds.), Plenum Press, New York 1980; and Antibodies: A Laboratory Manual, Harlow and Land (eds.), Cold Spring Harbor Laboratory Press, Cold Spring Harbor, N.Y., 1988; see also U.S. Pat. No. 4,411,993). One exemplary protocol for making monoclonal antibodies is that described in WO 98/48017. In brief, BALB-C mice are immunized at 0, 2 and 6 weeks with 10 μg of the antigenic LIR polypeptide. The primary immunization utilizes TITERMAX adjuvant, from Vaxcell, Inc., and subsequent immunizations use incomplete Freund's adjuvant (IFA). At 11 weeks, the mice are boosted by intravenous administration of 34 μg the antigenic protein in PBS. Three days after the intravenous boost, splenocytes are harvested and fused with an Ag8.653 myeloma fusion partner using 50% aqueous PEG 1500 solution. To screen the hybridoma cells, supernatants from individual colonies of hybridoma cells are screened by ELISA against COS-1 cells that have been transfected with the LIR against which antibodies are being raised. For this screening, 2×10³ transfected COS-1 cells in PBS are added to individual wells of a polystyrene 96-well microtiter plate and the cells dried to'the plate as the platecoat antigen. Positive supernatants are subsequently confined by FACS analysis and RIP using LIR-transfected COS-1 cells. Hybridomas are cloned and followed using the same assays. Monoclonal cultures are expanded and supernatants purified by affinity chromatography using BioRad Protein A agarose. Variations of the above procedures are known in the art, and if desired may be used to make or screen the hybridomas.

The monoclonal antibodies of the present invention also include humanized versions of murine or rat monoclonal antibodies. Such humanized antibodies can be prepared by known techniques and offer the advantage of reduced immunogenicity when the antibodies are administered to humans. In one embodiment, a humanized monoclonal antibody comprises the variable region of a mouse or rat antibody (or just the antigen binding site thereof) and a constant region derived from a human antibody. Alternatively, a humanized antibody fragment can comprise the antigen binding site of a murine or rat monoclonal antibody and a variable region fragment (lacking the antigen-binding site) as well as a constant region derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Useful techniques for humanizing antibodies are also discussed in U.S. Pat. No. 6,054,297. Procedures to generate antibodies transgenically can be found in GB 2,272,440, U.S. Pat. Nos. 5,569,825 and 5,545,806, and related patents. Preferably, for use in humans, the antibodies are human or humanized; techniques for creating such human or humanized antibodies are also well known and are commercially available from, for example, Medarex Inc. (Princeton, N.J.) and Abgenix Inc. (Fremont, Calif.). In another preferred embodiment, fully human antibodies for use in humans are produced by screening a phage display library of human antibody variable domains (Vaughan et al., 1998, Nat Biotechnol. 16(6):535-539; and U.S. Pat. No. 5,969,108).

Screening procedures are employed to determine whether a LIR-specific antibody is agonistic or antagonistic. Agonistic antibodies specific for LIR-2 or LIR-3 are useful as therapeutic agents to treat RA and can be identified, for example, by their ability to downregulate calcium flux or tyrosine phosphorylation in activated monocytes that are expressing LIR-2 or LIR-3. For such assays, monocytes are activated and calcium flux measured as described above in the presence or absence of the LIR-2 specific or LIR-3 specific antibody that is being screened. The activated monocytes are exposed during the analysis to the putative agonistic antibody. Antagonistic antibodies against LIR-7 useful as therapeutic agents for RA can be identified by their ability to bind to LIR-7 and not trigger calcium flux in monocytes.

In one aspect of the invention, the antigenic stimulus used to raise LIR-specific antibodies for use as a therapeutic agent is a fusion protein comprising the Fc portion of human IgG and the extracellular region of the target LIR. One suitable Fc polypeptide for this purpose is the native Fc region polypeptide derived from a human IgG1 that is described in PCT patent application WO 93/10151, which is hereby incorporated herein by reference. Another useful Fc polypeptide for constructing fusion proteins is the Fc mutein described in U.S. Pat. No. 5,457,035. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. This mutein Fc exhibits reduced affinity for immunoglobulin receptors.

One suitable means of making a LIR:Fc fusion protein is that described in WO 98/48017. To apply this method, cDNA encoding all or part of the extracellular region of LIR-2, LIR-3 or LIR-7 is fused with a DNA encoding a human IgG Fc region. For making a LIR-2:Fc fusion, one may use the entire extracellular region (amino acids 1-458 of SEQ ID NO:2) or a portion thereof that is capable of binding MHC I. The cDNA encoding the extracellular region of the LIR is obtained by using PCR primers that flank the nucleotide sequences that encode the extracellular region. For this purpose, full-length LIR cDNA may be used as the template for PCR, such as, for example, cDNAs having a nucleotide sequence as shown in SEQ ID NO:1, NO:3 or NO:5. In one embodiment of the invention, the primers are synthesized with Sal I and Not II restriction sites inserted at the 5′ and 3′ termini so that the amplification product introduces Sal I and Not II restriction sites at the 5′ and 3′ ends, respectively, of the amplified DNA fragment. These restriction sites facilitate attachment of the amplified DNA into an expression vector. Alternatively, other restriction sites may be inserted at the ends of the amplified DNA. In a preferred embodiment of the invention, to prepare a vector construct for expressing the fusion proteins, DNA encoding the mutein human Fc region of IgG1 is ligated to DNA encoding the extracellular region of LIR-2, LIR-3 or LIR-7. In one embodiment of the invention, this is done such that the LIR extracellular region is located at the amino terminus of the fusion protein. The fusion DNA constructs are then ligated into a suitable expression vector, such as PDC409, and expressed in a suitable host cell, such as CV1-EBNA or COS cells (ATCC CTL-1650). The monkey cell line COS-1 may be used to confirm the expression of the fusion protein. To purify the fusion proteins, supernatant from COS-1 cultures is clarified by centrifugation and purified, for example, by using the BioCad system and the POROS 20A column from PerSeptive Biosystems, or by other comparable methods known to those skilled in the art. The pooled eluted protein may be analyzed using SDS polyacrylamide gel electrophoresis with silver staining to confirm expression and to verify that the expressed recombinant protein has the expected molecular weight.

Concurrent Treatments

Provided herein are combination treatments in which one or more modulators of LIR-2, LIR-3 or LIR-7 are administered concurrently with one or more other medications used to treat RA. In such methods of treatment, the LIR modulators may be administered concurrently with one, two, three or more other medications used to treat RA. The additional medications may be administered simultaneously or alternately with the LIR modulators. Alternatively, LIR modulators may be administered intermittently against a background of continuous treatment with some other agent being used to treat RA, such as for example an antagonist of tumor necrosis factor α (TNFα) or an antagonist of IL-1. The LIR modulators and other RA treatments may be administered simultaneously, alternately or sequentially, and the frequency of their administration may be the same or different.

TNFα antagonists suitable for concurrent use with LIR modulators in treating RA include antibodies against TNFα. Such antibodies include humanized and fully human monoclonal antibodies. An exemplary humanized antibody for concurrent administration with a LIR modulator is a chimeric IgG1κ monoclonal antibody called infliximab, which is sold by Centocor as REMICADE®. Infliximab is composed of human constant and murine variable regions, and binds specifically to human TNFα. Other suitable anti-TNFα antibodies include the humanized antibodies D2E7 and CDP571, and the antibodies described in EP 0 516 785 B1, U.S. Pat. No. 5,656,272, EP 0 492 448 A1.

The LIR modulator may be administered concomitantly with various kinds of agents that are used to treat RA, including inhibitors of IL-1, antibodies directed against T-cell surface proteins and antisense oligonucleotides or ribozymes that interfere with the translation of TNFα, a TNFα receptor, or an enzyme in the metabolic pathways for the synthesis of TNFα. Suitable antisense oligonucleotides for concurrent administration with LIR modulators include those described in U.S. Pat. No. 6,228,642. Also suitable for concurrent administration with LIR modulators are the peptide TNFα inhibitors disclosed in U.S. Pat. No. 5,641,751 and U.S. Pat. No. 5,519,000, and the D-amino acid-containing peptides described in U.S. Pat. No. 5,753,628. In addition, the conditions described herein may be treated with inhibitors of TNFα converting enzyme.

Preferred combinations include administration of LIR modulators concurrently with a TNFα inhibitor that is a soluble form of a TNF receptor (sTNFR). sTNFRs may include monomers, fusion proteins (also called “chimeric proteins), dimers, trimers or higher order multimers. In certain embodiments of the invention, the sTNFR derivative is one that mimics the 75 kDa TNFR or the 55 kDa TNFR and that binds to TNFα in the patient's body. The sTNFR mimics of the present invention may be derived from TNFRs p55 or p75 or fragments thereof. TNFRs other than p55 and p75 also are useful for deriving soluble compounds for treating the various medical disorders described herein, such for example the TNFR that is described in WO 99/04001. sTNFR molecules used to construct TNFR mimics include, for example, analogs or fragments of native TNFRs having at least 20 amino acids, that lack the transmembrane region of the native TNFR, and that are capable of binding TNFα. Binding of sTNFRs to TNFα can be assayed using ELISA or any other convenient assay.

The sTNFR polypeptides or fragments of the invention may be fused with a second polypeptide to form a chimeric protein. The second polypeptide may promote the spontaneous formation by the chimeric protein of a dimer, trimer or higher order multimer that is capable of binding a TNFα or a LTα molecule and preventing it from binding to cell-bound receptors. Chimeric proteins used as antagonists include, for example, molecules derived from the constant region of an antibody molecule and the extracellular portion of a TNFR. Such molecules are referred to herein as TNFR-Ig fusion proteins. A preferred TNFR-Ig fusion protein suitable for treating diseases in humans and other mammals is recombinant TNFR:Fc, a term which as used herein refers to “etanercept,” which is a dimer of two molecules of the extracellular portion of the p75 TNFα receptor, each molecule consisting of a 235 amino acid TNFR-derived polypeptide that is fused to a 232 amino acid Fc portion of human IgG₁. Etanercept is currently sold by Immunex Corporation under the trade name ENBREL.® Also encompassed by the invention are treatments combining LIR modulators with a compound that comprises the extracellular portion of the 55 kDa TNFR fused to the Fc portion of IgG, as well as compositions and combinations containing such therapeutic agents. In addition, suitable TNF inhibitors may be derived from the extracellular regions of TNFα receptor molecules other than the p55 and p75 TNFRs, such as for example the TNFR described in WO 99/04001, including TNFR-Ig's derived from this TNFR.

In addition, the subject LIR modulators can be administered to RA patients concurrently with agents that diminish bone destruction in arthritic joints, including, for example, soluble forms of RANK, such as RANK:Fc, or osteoprotegerin (see U.S. Pat. No. 6,017,729 and WO 98/46751).

Other drugs suitable for concurrent administration with a LIR modulator include analgesics including but not limited to acetaminophen, codeine, propoxyphene napsylate, oxycodone hydrochloride, hydrocodone bitartrate and tramadol. In addition, LIR modulator may be administered concomitantly with a disease-modifying anti-rheumatic drug (DMARD), including but not limited to methotrexate, sulfasalazine, gold salts, azathioprine, cyclosporine, antimalarials, steroids (e.g., prednisone) and colchicine.

Anti-inflammatories may also be coadministered with the LIR modulator. Such anti-inflammatories include but are not limited to: aspirin; ibuprofen; indomethacin; celecoxib; rofecoxib; ketorolac; nambumetone; piroxicam; naproxen; oxaprozin; sulindac; ketoprofen; diclofenac; and other COX-1 and COX-2 inhibitors, salicylic acid derivatives, propionic acid derivatives, acetic acid derivatives, fenamic acid derivatives, carboxylic acid derivatives, butyric acid derivatives, oxicams, pyrazoles and pyrazolones, including newly developed antiinflammatories that are effective for treating arthritic joints.

IL-1 inhibitors suitable for concurrent use with LIR modulators include receptor-binding peptide fragments of IL-1, antibodies directed against IL-1 or IL-1 beta or IL-1 receptor type I, and recombinant proteins comprising all or portions of receptors for IL-1 or modified variants thereof, including genetically-modified muteins, multimeric forms and sustained-release formulations. Particular antagonists include IL-1ra polypeptides, IL-1 beta converting enzyme (ICE) inhibitors, antagonistic type I IL-1 receptor antibodies, IL-1 binding forms of type I IL-1 receptor and type II IL-1 receptor, antibodies to IL-1, including IL-1 alpha and IL-1 beta and other IL-1 family members, and therapeutics known as IL-1 traps. IL-1ra polypeptides include the forms of IL-1ra described in U.S. Pat. No. 5,075,222 and modified forms and variants including those described in U.S. Pat. No. 5,922,573, WO 91/17184, WO 92/16221, and WO 96/09323, all of which are. IL-1 beta converting enzyme (ICE) inhibitors include peptidyl and small molecule ICE inhibitors including those described in PCT patent applications WO 91/15577; WO 93/05071; WO 93/09135; WO 93/14777 and WO 93/16710; and European patent application 0 547 699. Non-peptidyl compounds include those described in PCT patent application WO 95/26958, U.S. Pat. No. 5,552,400, U.S. Pat. No. 6,121,266, Dolle et al., J. Med. Chem., 39:2438-2440 (1996). Additional ICE inhibitors are described in U.S. Pat. Nos. 6,162,790, 6,204,261, 6,136,787, 6,103,711, 6,025,147, 6,008,217, 5,973,111, 5,874,424, 5,847,135, 5,843,904, 5,756,466, 5,656,627, 5,716,929.

IL-1 binding forms of type I IL-1 receptor and type II IL-1 receptor suitable for concurrent administration with LIR modulators are described in U.S. Pat. Nos. 4,968,607, 4,968,607, 5,081,228, Re 35,450, U.S. Pat. Nos. 5,319,071, and 5,350,683. IL-1 traps are described in WO 018932.

Suitable IL-1 antagonists suitable for concurrent administration with LIR modulators further include chimeric proteins that include portions of both an antibody molecule and an IL-1 antagonist molecule. Such chimeric molecules may form monomers, dimers or higher order multimers. Other suitable IL-1 antagonists include peptides derived from IL-1 that are capable of binding competitively to the IL-1 signaling receptor, IL-1 R type I.

In addition, LIR modulators may be used to treat RA in conjunction with small molecules such as thalidomide or thalidomide analogs, pentoxifylline, matrix metalloproteinase (MMP) inhibitors or other small molecules. Suitable MMP inhibitors include, for example, those described in U.S. Pat. Nos. 5,883,131, 5,863,949 and 5,861,510 as well as the mercapto alkyl peptidyl compounds described in U.S. Pat. No. 5,872,146. Other small molecules suitable for concurrent administration are molecules capable of reducing TNFα production, including those described in U.S. Pat. Nos. 5,508,300, 5,596,013 and 5,563,143, any of which can be administered in combination with LIR modulators. Additional small molecules useful for treating RA in conjunction with LIR modulators include the MMP inhibitors that are described in U.S. Pat. No. 5,747,514 or U.S. Pat. No. 5,691,382, as well as the hydroxamic acid derivatives described in U.S. Pat. No. 5,821,262, and small molecules that inhibit phosphodiesterase IV and TNFα production, such as substituted oxime derivatives (WO 96/00215), quinoline sulfonamides (U.S. Pat. No. 5,834,485), aryl furan derivatives (WO 99/18095) and heterobicyclic derivatives (WO 96/01825; GB 2 291 422 A), thiazole derivatives that suppress TNFα and IFNγ (WO 99/15524), as well as xanthine derivatives that suppress TNFα and other proinflammatory cytokines (see, for example, U.S. Pat. No. 5,118,500, U.S. Pat. No. 5,096,906 and U.S. Pat. No. 5,196,430).

Pharmaceutical Preparations

Pharmaceutical compositions comprising an effective amount of a LIR modulator of the present invention (from whatever source derived, including without limitation from recombinant and non-recombinant sources), in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. Therapeutic agents according to the invention can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, LIR modulators for pharmaceutical compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323. Such compositions will influence the physical state, solubility, stability, rate of in vivo release, and rate of in vivo clearance, and are thus chosen according to the intended application, so that the characteristics of the carrier will depend on the selected route of administration.

In one preferred embodiment of the invention, sustained-release forms of LIR modulating polypeptides are used. Sustained-release forms suitable for use in the disclosed methods include, but are not limited to, LIR modulators that are encapsulated in a slowly-dissolving biocompatible polymer (such as the alginate microparticles described in U.S. Pat. No. 6,036,978), admixed with such a polymer (including topically applied hydrogels), and/or encased in a biocompatible semi-permeable implant. Also included are formulations of microparticles suitable for injection into which the therapeutic agent or agents have been incorporated. Included also are therapeutic LIR modulators that have been modified to increase their half-life in the blood. For example, the LIR modulator may be conjugated with polyethylene glycol for injection using known methods.

Regimen for Administration

As used herein, the phrase “administering a therapeutically effective amount” of an agent that antagonizes LIR-7 or agonizes LIR-2 or LIR-3 means that the patient is treated with the therapeutic agent in an amount and for a time sufficient to induce an improvement, preferably a sustained improvement, in at least one indicator that reflects the severity of the disorder. An improvement is considered “sustained” if the patient exhibits the improvement on at least two occasions separated by one or more days, or more preferably, by one or more weeks. The degree of improvement is determined based on signs or symptoms, and determinations may also employ questionnaires that are administered to the patient, such as quality-of-life questionnaires. Various indicators that reflect the extent of the patient's illness may be assessed for determining whether the amount and time of the treatment is sufficient. The baseline value for the chosen indicator or indicators is established by examination of the patient prior to administration of the first dose of the therapeutic agent. Preferably, the baseline examination is done within about 60 days of administering the first dose, and may be performed at any time up to the same day as the first dose. Improvement is induced by administering therapeutic agents according to the invention until the patient manifests an improvement over baseline for the chosen indicator or indicators. In this context, “baseline” refers to the measurement of the chosen indicator prior to the first dose in the patient being treated.

In one embodiment of the invention, a sufficient amount and time of treatment for RA will occur when the treatment has induced an improvement according to the American College of Rheumatology (ACR) criteria as modified by Felson et al. (Felson et al., Arthritis Rheum 6:727-735, 1995). When ACR criteria are used, the treatment is considered to be sufficient when the patient has improved by at least 20% (ACR20) or by at least 50% (ACR50) in both tender joint count (ca. 78 joints assessed) and swollen joint count (ca. 76 joints assessed), and also shows an improvement in three of the following five: 1) subject pain assessment; 2) subject global assessment; 3) physician global assessment; 4) subject self-assessed disability; 5) acute-phase reactant (Westergreen erythrocyte sedimentation rate or C-reactive protein level). Of the preceding five criteria, the first four are scored on a Likert scale. Subject and global assessments are determined based on the overall status of the patient's disease.

In another embodiment of the invention, sufficiency of treatment is assessed by patient self-assessment or physician assessment. Patient self-assessment or physician assessment may be measured, for example, on a subjective numerical scale in which one extreme of the scale represents “no disease,” and the other extreme indicates “severe disease” (i.e., a “Likert” scale). Either extreme of the scale may be used to represent “no disease.” Such a scale can have any desired range of numerical values, e.g., 0-3, 0-4, 0-5, 0-6, 0-7, etc., and provides a basis for comparison to the patient's condition at baseline. A 0-3 point system, for example, could involve the following categories: 0=no disease; 1=mild disease; 2=moderate disease; 3=severe disease. In this example, a patient would be regarded as “improved” if their score decreased by one category. As used herein, the term “Likert scale” is understood to include visual analog scales (VAS), in which a patient or physician circles a number that they feel best represents the patient's status with respect to the parameter being measured. If a patient's score worsens as compared with their baseline score, the change in their Likert score is assigned a negative value.

Suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. A dose may be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of therapeutic agent of the present invention with which to treat each individual patient, and may modulate the does and frequency of administration in accord with an individual patient's needs.

Any efficacious route of administration may be used to therapeutically administer an LIR modulator for the subject methods of treatment. If injected, the LIR agonist or antagonist can be administered, for example, via intra-articular, intravenous, intramuscular, intralesional, intraperitoneal or subcutaneous routes by bolus injection or by continuous infusion. Other suitable means of administration include sustained release from implants, aerosol inhalation, eyedrops, oral preparations, including pills, syrups, lozenges or chewing gum, and topical preparations such as lotions, gels, sprays, ointments or other suitable techniques. Alternatively, proteinaceous LIR modulators may be administered by implanting cultured cells that express the protein, for example, by implanting cells that express the modulating protein, i.e., an antagonist of LIR-7, an agonist of LIR-2 or an agonist of LIR-3. In one embodiment, the patient's own cells are induced to produce the LIR modulator by transfection in vivo or ex vivo with a DNA that encodes the modulator. This DNA can be introduced into the patient's cells, for example, by injecting naked DNA or liposome-encapsulated DNA that encodes the modulator, by infection with a viral vector expressing the DNA, or by other means known in the art. When the LIR modulator is administered in combination with one or more other biologically active compounds, these may be administered by the same or by different routes, and may be administered simultaneously, separately or sequentially.

Pharmaceutical compositions for injection comprising proteins that are modulators of LIR-2, LIR-3 or LIR-7 should contain a dose of about 0.01 ng to about 100 mg (preferably about 0.1 ng to about 10 mg, more preferably about 0.1 microgram to about 1 mg) of the therapeutic polypeptide per kg body weight. In one embodiment of the invention, such compositions are administered one time per month to treat the various medical disorders disclosed herein, in another embodiment are administered at least one time per week, and in another embodiment are administered at least two or more times per week.

When the route of administration is injection or intravenous infusion, the effective amount of LIR modulators per adult dose may be calculated based on body surface area. A preferred dose range for a therapeutic antibody or other therapeutic agent comprising a protein is 1-20 mg/m², and more preferably 5-12 mg/m². Alternatively, a flat dose may be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, a medical disorder is treated by administering a preparation acceptable for injection containing therapeutic polypeptides at a flat dose containing 1, 5, 10, 25 or 50 mg. The therapeutic agent may be administered repeatedly at a frequency of once every eight weeks, once every seven weeks, once every six weeks, once every five weeks, once every four weeks, once every three weeks, once every two weeks, once every week, two times per week, three times per week, four times per week, five times per week, six times per week or daily. If a route of administration other than injection is used, the dose is appropriately adjusted in accord with standard medical practices.

If an antibody is used as the LIR-7 antagonist or as the LIR-2 or LIR-3 agonist, one preferred dose range is 1-10 mg/kg, and another preferred dose range is 0.75 to 7.5 mg/kg of body weight. Humanized antibodies are preferred, that is, antibodies in which only the antigen-binding portion of the antibody molecule is derived from a non-human source. Such antibodies may be injected subcutaneously, intramuscularly or administered intravenously, and may be used concurrently with methotrexate to reduce the development of host antibodies against the therapeutic agent.

The duration of the treatment may vary, depending on the patient's response. In many instances, an improvement in a patient's condition will be obtained by injecting the therapeutic dose over a period of at least three weeks, though treatment for longer periods may be necessary to induce the desired degree of improvement. The regimen may be continued indefinitely, with adjustments being made to dose and frequency if such are deemed necessary by the patient's physician. For pediatric patients (age 4-17), one suitable regimen involves the subcutaneous injection of 0.4 mg/kg, up to a maximum dose of 25 mg of a polypeptide LIR modulator, administered by subcutaneous injection one or more times per week.

In treating RA, which is a chronic condition, improvement in the patient's condition is obtained by repeatedly administering this medicament over a period of at least a week or more, or more preferably for a month or more, or for one, two, or three months or longer, or indefinitely. Treatment may be continued indefinitely at the same dose and frequency as the initial month of treatment, or at a reduced dose or frequency, or may be administered intermittently in conjunction with some other treatment that is being used as the primary treatment for a given patient. If the dose or frequency of administration is reduced or discontinued, it later may be resumed at the original level if symptoms should worsen. Such determinations are made by the patient's physician, in accord with standard medical practices.

It is understood that the response by individual patients to the aforementioned medications may vary, and the most efficacious combination of drugs and dosing regimens for each patient will be determined by his or her physician.

The following example is offered by way of illustration, and not by way of limitation. Those skilled in the art will recognize that variations of the invention embodied in this example can be made, especially in light of the teachings of the various references cited herein.

### EXAMPLE

This study compared the in vivo expression and cellular distribution of several LIRs in the synovia of patients having RA, osteoarthritis (OA) and normal patients. OA synovium was included in this study because, in contrast to RA, inflammatory reactions in the synovial tissue in OA occur in the absence of pannus formation and tissue invasion (Ehrlich et al., In: Moskowitz et al., Eds., Osteoarthritis: diagnosis and management, Philadelphia, W. B. Saunders, 199-211 (1984); Dieppe et al., In: Moskowitz et al., Eds, Osteoarthritis: diagnosis and medical/surgical management. 2nd ed., Philadelphia, W. B. Saunders, 399-412 (1992)). Using immunohistochemistry, the expression of inhibitory and activating LIRs was studied in the synovium of six RA patients, three osteoarthritis patients, and three control subjects. Staining with specific antibodies was used to detect expression of LIR-2, LIR-3 and LIR-7, and the staining of serial sections with cell-lineage specific antibodies was used to evaluate the cellular localization of expressed LIRs.

The expression of two inhibitory LIR polypeptides, LIR-2 and LIR-3, and one activating LIR polypeptide, LIR-7, were examined in this set of experiments. In general, these three LIRs have a relatively restricted expression on cells of myeloid origin, which cells are important sources of cytokines and proteases in RA. LIR-2 is known to recognize MHC class I molecules, which are widely distributed in human tissues.

Six patients with a history of RA ranging from two to fourteen years and three patients with a history of OA ranging from three to thirteen years, underwent excision of synovial tissue from the knee joint under general anesthesia. Normal synovial tissue was obtained from three subjects during re-constructive knee surgery for traumatic meniscus rupture. Synovial tissue was embedded in OCT compound, snap frozen in liquid nitrogen. (Tissue-Tek, Miles, Elkhart, Ind.) and sectioned at 2-4 μm for histopathological analysis and immunohistochemical studies.

Specific mouse IgG1 monoclonal antibodies against LIR-2, LIR-3 and LIR-7 were generated in BALB/c mice by immunization with LIR/Fc fusion proteins containing the LIR extracellular domains fused to the Fc region of human IgG1 as described (see Cosman et al., Immunity 7:273-282 (1997)). The antibodies were screened for specific immunoreactivity by ELISA against a panel of LIR/Fc fusion proteins and by FACS analysis using COS-1 cells transfected with full-length LIR cDNAs. Irrelevant mouse IgG1 antibody was used as a negative control (Biosource International, Camarillo, Calif.).

These antibodies were used in a three-step alkaline phosphatase staining technique as described in Tedla et al., Am J Pathol 148(5):1367-73 (1996), which is hereby incorporated by reference in its entirety. In brief, acetone-fixed sections were equilibrated with TRIS-buffered saline (TBS) and blocked with neat horse serum for 20 minutes at room temperature. Sections were then incubated with 5 μg/ml primary antibodies overnight at 4° C. After 4 washes with TBS, sections were incubated with biotylinated horse anti-mouse IgG (Vector laboratories, Burlingame, Calif.) for one hour at room temperature. After 4 washes with TBS, the sections were incubated with streptavidin-alkaline phosphatase conjugate (Vector laboratories) for 45 minutes at room temperature. Immunoreactivity was detected using a calorimetric alkaline phosphatase substrate (vector red, Vector laboratories) and brief counter-staining with hematoxylin. Optimal conditions for use of each anti-LIR antibody were initially defined using a panel of normal tissues likely to contain LIR-expressing cells, including skin, thymus, lymph nodes and spleen cells.

Sections adjacent to those analyzed with anti-LIR antibodies were also analyzed in order to determine the specific cell types that are immunoreactive with antibodies specific for the LIRs. This was done using the methods described in Tedla et al., Cytokine 11 (7):531-40 (1999), which is hereby incorporated by reference, and Tedla et al., 1996. This effort employed antibodies to detect macrophages (mouse IgG1 anti-CD68), T cells (rabbit polyclonal anti-CD3), endothelial cells (mouse IgG1 anti-Von-Willebrand factor), neutrophil cathepsin G (rabbit polyclonal) and mast cell tryptase (mouse IgG1). These antibodies were purchased from DAKO (Glostrup, Denmark). The staining procedure used was essentially as described above for staining with anti-LIR antibodies. In addition to the immunohistochemical staining with specific antibodies, a standard hematoxylin and eosin stain was used to evaluate the quality and histology of each section.

The tissue sections from the immunohistochemical studies were evaluated by counting the cells seen in contiguous fields across the whole section. In brief, an average of 18 fields at magnification of 250× was selected per section in a systematic sampling procedure. After ensuring that control sections stained with isotype control antibody exhibited no significant immunoreactivity, the number of positive cells (red staining) per field was enumerated. Although significant regional variation in staining was observed, the median count for the whole section was reported as a conservative measure of the staining for each antibody.

The observed histological features of the synovial tissue samples and expression of the three LIRs are summarized in Table 1. Sections from two RA patients (RA1 and RA2) who had a relatively short duration of illness (2-5 years) showed extensive infiltration with inflammatory cells, including CD68-positive macrophages, cathepsin G-positive neutrophils, a moderate number of tryptase-positive mast cells and clusters of CD3-positive T cells. In the remaining four RA patients, who had been afflicted for 8-14 years, there were varying degrees of inflammatory cell infiltration and in these patients, tissue fibrosis was evident. In sections from two of the patients with OA, there was significant macrophage infiltration with limited numbers of T cells and mast cells (Table 1). The third OA patient had extensive fibrosis with macrophages at the outer edges of the synovial membrane. Few or no inflammatory cells were observed in the sections obtained from normal subjects.

As shown in Table 1, there was extensive expression of LIR-2 and LIR-7 in sections obtained from three RA patients with early to intermediate duration of illness (patients RA1, RA2 and RA3). LIR-2 was co-localized with the LIR-7 on neutrophils and macrophages infiltrating the rheumatoid synovium. The expression of LIR-2 and LIR-7 was especially marked in early rheumatoid disease (Table 1), but was extremely limited for patients with a long duration of RA (8 years or longer). With increasing duration of RA, the synovial tissue showed more fibrotic changes and the number of cells expressing LIR-2 and LIR-7 dramatically decreased. Negligible expression of both the activating and inhibitory LIRs was found in synovial tissues obtained from patients with OA. No LIR expression was detected in synovial tissue from normal donors.

LIR-3 was expressed in infiltrating macrophages in the rheumatoid synovium of two out of three early RA patients, but not in late RA patients. No expression of LIR-2, LIR-3 or LIR-7 was detected in control tissues obtained from normal subjects.

Table 2 presents the results obtained by staining serial sections of synovial tissue samples with LIR-specific antibodies and specific for macrophages, endothelial cells, neutrophils, mast cells and CD3+ T cells. No LIRs were detected on CD3+ T cells. As shown in Table 2, in contrast to the restriction of LIR-2 and LIR-3 expression to inflammatory leukocytes, LIR-7 was variably expressed on neutrophils, macrophages, mast cells, fibroblast-like cells (as determined by cell morphology), and endothelial cells in synovium of RA patients. The cellular distribution of LIR-2 and LIR-7 differed among RA patients, reflecting differences in the nature of the inflammatory cell infiltrate. Neutrophils were the major cellular source of LIR-2 in patient RA1, but macrophages were the major cellular source of LIR-2 in patient RA2. Among the RA patients in general, expression of LIR-7 was somewhat less than that of LIR-2 and the cellular distribution of LIR-7 was wider than that of LIR-2. In patient RA1, LIR-7 was expressed by neutrophils, macrophages, mast cells and endothelial cells, and in patient RA2, by macrophages, endothelial cells and fibroblast-like synoviocytes. The cellular sources of LIR-2 and LIR-7 in all remaining RA patients were macrophages, and to a lesser extent endothelial cells. The limited expression of LIR-2 that was observed in OA was found mainly on CD68+macrophages. In patients with RA, LIR-3 was exclusively expressed by macrophages and fibroblast-like synoviocytes. Isotype matched negative control antibodies did not yield immunostaining in any of the patients.

The results presented above reveal that synovium from patients with early RA showed elevated expression of the inhibitory LIR-2, which is capable of recognizing and associating with MHC class I molecules. Elevated expression of LIR-3 also was observed in RA patients. Extensive expression of the LIR-7, which has the capacity to activate cells, was observed also on macrophages and neutrophils from RA patients. In addition, some LIR-7 expression was observed on mast cells and endothelial cells. Little LIR expression was observed in synovium from patients with OA, in control subjects, or in two patients with long-standing RA that was accompanied by fibrosis. Thus, these results suggest that LIRs may regulate protease and cytokine expression in the inflammatory infiltrate in RA and thereby contribute to the process of pannus formation and joint destruction.

TABLE 1
Expression of Leukocyte immunoglobulin-like receptors in RA, OA and normal synovium

| | | | Immunohistochemical | | |
|---|---|---|---|---|---|
| | | | expression of | | |
| | | | LIRs (median cell | | |
| | Duration of illness | | count/HPF) | | |
| Subjects | (years) | Histology | LIR-2 | LIR-3 | LIR-7 |
| Rheumatoid arthritis | | | | | |
| RA1 | 2 | Extensive neutrophil infiltration and | 39 | 6 | 18 |
| | | moderate numbers of macrophages and | | | |
| | | mast cells. | | | |
| RA2 | 5 | Widespread macrophage infiltration and | 25 | 1 | 13 |
| | | small areas of lymphocyte aggregation | | | |
| RA3 | 8 | Macrophage and lymphocyte aggregation. | 15 | 7 | 8.5 |
| | | Moderate degree of fibrosis with mast cell | | | |
| | | infiltration. | | | |
| RA4 | 8 | Extensive fibrosis and endothelial | 5 | 2 | 3 |
| | | proliferation with some areas of CD68+ | | | |
| | | macrophage infiltration | | | |
| RA5 | 10 | Extensive fibrosis with small numbers of | 0 | 0.5 | 0.5 |
| | | macrophages | | | |
| RA6 | 14 | Extensive fibrosis with small numbers of | 0.5 | 0.5 | 0.5 |
| | | macrophages | | | |
| Osteoarthritis | | | | | |
| OA1 | 3-5 | Moderate macrophage and lymphocyte | 0.5 | 1 | 0 |
| | | infiltration | | | |
| OA2 | 9 | Moderate macrophage and lymphocyte | 0 | 2.5 | 0 |
| | | infiltration. Limited numbers of mast cells | | | |
| OA3 | 13 | Extensive fibrosis | 2 | 7 | 1 |
| Controls | | | | | |
| N1 | — | | 0 | 0 | 0 |
| N2 | — | | 0 | 0 | 0 |
| N3 | — | | 0 | 0 | 0 |

TABLE 2
The cellular sources of LIR-2, LIR-3, and LIR-7 in synovium
from patients with rheumatoid arthritis and osteoarthritis.

| Subjects | LIR-2 | LIR-3 | LIR-7 |
|---|---|---|---|
| Rheumatoid arthritis | | | |
| RA1 | Neutrophils | Macrophages | Neutrophils, macrophages, |
| | | | mast cells, endothelial cells |
| RA2 | Macrophages | Macrophages and | Macrophages, endothelial |
| | | fibroblast-like cells | cells, fibroblast-like cells |
| RA3 | Macrophages, endothelial | Macrophages and | Macrophages, mast cells, |
| | cells | fibroblast-like cells | endothelial cells |
| RA4 | Macrophages | Fibroblast-like cells, | Macrophages, endothelial |
| | | macrophages | cells |
| RA5 | ND | Fibroblast-like cells | Endothelial cells |
| RA6 | ND | ND | Fibroblast-like cells |
| Osteoarthritis | | | |
| OA1 | Macrophages | Macrophages | Macrophages |
| OA2 | ND | Macrophages | ND |
| OA3 | Fibroblast-like cells | Fibroblast-like cells | Endothelial cells |

ND, not done due to limited LIR expression.

1. A method for treating a patient having rheumatoid arthritis comprising administering to said patient a therapeutically effective amount of one or more agents that modulate the expression or function of LIR-2 and/or LIR-3 and/or LIR-7.

2. A method according to claim 1, wherein said treatment comprises administering to said patient an agent that antagonizes the activity of LIR-7.

3. A method according to claim 1, wherein said treatment comprises administering to said patient an agent that agonizes LIR-2.

4. A method according to claim 1, wherein said treatment comprises administering to said patient an agent that agonizes LIR-3.

5. A method according to claim 1, wherein said treatment comprises administering concurrently to said patient an agent a combination selected from the group consisting of: an agent that agonizes LIR-3 and an agent that antagonizes LIR-7; an agent that agonizes LIR-2 and an agent that antagonizes LIR-7; an agent that agonizes LIR-2 and an agent that agonizes LIR-2; an agent that agonizes LIR-2, an agent that agonizes LIR-2 and an agent that antagonizes LIR-7.

6. A method according to claim 2, wherein said antagonist of LIR-7 is an antagonistic antibody that is specifically immunoreactive with LIR-7.

7. A method according to claim 3, wherein said agonist of LIR-2 is an agonistic antibody that is specifically immunoreactive with LIR-2.

8. A method according to claim 4, wherein said agonist of LIR-3 is an agonistic antibody that is specifically immunoreactive with LIR-3.

9. A method according to claim 1, wherein said patient is concurrently treated with at least one additional drug selected from the group consisting of a TNFα antagonist, an IL-1 antagonist, an antibody against CD4, a non-steroidal anti-inflammatory drug, an analgesic and a disease-modifying anti-rheumatic drug.

10. A method for treating a patient having rheumatoid arthritis comprising administering to said patient a therapeutically effective amount of one or more agents selected from the group consisting of an agonistic monoclonal antibody that is specifically immunoreactive with LIR-2, an agonistic monoclonal antibody that is specifically immunoreactive with LIR-3 and an antagonistic monoclonal antibody that is specifically immunoreactive with LIR-7.


SEQUENCE LISTING (6 sequences)

SEQ ID NO: 1 - DNA, 2221 bp, Homo sapiens
  CDS (184)..(1977)
gctcactgccacacgcagctcagcctgggcggcacagccagatgcgagatgcgtctctgctgatctgagtctgcctgcagcatggacctgggtcttccctgaagcatctccagggctggagggacgactgccatgcaccgagggctcatccatccgcagagcagggcagtgggaggagacgccatgacccccatcgtcacagtcctgatctgtctcgggctgagtctgggccccaggacccacgtgcagacagggaccatccccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagatgaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcgtggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcatacaacctctcctccgagtggtcggcccccagcgaccccctggacatcctgatcacaggacagatccatggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctgcaggccctgaggaccagcccctcacccccactgggtcggatccccaaagtggtctgggaaggcacctgggggttgtgatcggcatcttggtggccgtcgtcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccactagcccggagggtacgcagactccacactcagtagaaggagactcaggactgctgaaggcacgggagctgcccccagtggacaccaatgaaccccagtcagcctggacccctaacaaagaccatgaggagatgctgggaactttgggactcacttgattctgcagtcgaaataactaatatccctacattttttaattaaagcaacagacttctcaataaaagcaggtcgtctcgttccaatct

SEQ ID NO: 2 - protein, 598 aa, Homo sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 3 - DNA, 2194 bp, Homo sapiens
  CDS (67)..(1959)
tctctgtcctgccagcactgagggctcatccctctgcagagcgcggggtcaccggaaggagacgccatgacgcccgccctcacagccctgctctgccttgggctgagtctgggccccaggacccgcgtgcaggcagggcccttccccaaacccaccctctgggctgagccaggctctgtgatcagctgggggagccccgtgaccatctggtgtcaggggagcctggaggcccaggagtaccaactggataaagagggaagcccagagcccttggacagaaataacccactggaacccaagaacaaggccagattctccatcccatccatgacacagcaccatgcagggagataccgctgccactattacagctctgcaggctggtcagagcccagcgaccccctggagctggtgatgacaggagcctatagcaaacccaccctctcagccctgcccagccctgtggtggcctcaggggggaatatgaccctccgatgtggctcacagaagagatatcaccattttgttctgatgaaggaaggagaacaccagctcccccggaccctggactcacagcagctccacagtggggggttccaggccctgttccctgtgggccccgtgaaccccagccacaggtggaggttcacatgctattactattatatgaacaccccccgggtgtggtcccaccccagtgaccccctggagattctgccctcaggcgtgtctaggaagccctccctcctgaccctgcagggccctgtcctggcccctgggcagagtctgaccctccagtgtggctctgatgtcggctacgacagatttgttctgtataaggagggggaacgtgacttcctccagcgccctggccagcagccccaggctgggctctcccaggccaacttcaccctgggccctgtgagcccctccaatgggggccagtacaggtgctacggtgcacacaacctctcctccgagtggtcggcccccagcgaccccctgaacatcctgatggcaggacagatctatgacaccgtctccctgtcagcacagccgggccccacagtggcctcaggagagaacgtgaccctgctgtgtcagtcatggtggcagtttgacactttccttctgaccaaagaaggggcagcccatcccccactgcgtctgagatcaatgtacggagctcataagtaccaggctgaattccccatgagtcctgtgacctcagcccacgcggggacctacaggtgctacggctcacgcagctccaacccctacctgctgtctcaccccagtgagcccctggagctcgtggtctcaggacactctggaggctccagcctcccacccacagggccgccctccacacctggtctgggaagatacctggaggttttgattggggtctcggtggccttcgtcctgctgctcttcctcctcctcttcctcctcctccgacgtcagcgtcacagcaaacacaggacatctgaccagagaaagactgatttccagcgtcctgcaggggctgcggagacagagcccaaggacaggggcctgctgaggaggtccagcccagctgctgacgtccaggaagaaaacctctatgctgccgtgaaggacacacagtctgaggacggggtggagctggacagtcagagcccacacgatgaagacccccacgcagtgacgtatgccccggtgaaacactccagtcctaggagagaaatggcctctcctccttccccactgtctggggaattcctggacacaaaggacagacaggcagaagaggacagacagatggacactgaggctgctgcatctgaagcctcccaggatgtgacctacgcccagctgcacagcttgacccttagacggaaggcaactgagcctcctccatcccaggaaggggaacctccagctgagcccagcatctacgccactctggccatccactagcccggggggtacgcagaccccacactcagcagaaggagactcaggactgctgaaggacgggagctgcccccagtggacaccagtgaaccccagtcagcctggacccctaacacagaccatgaggagacgctgggaacttgtgggactcacctgactcaaagatgactaatatcgtcccattttggaaataaagcaacagacttctcaagcaggtcgtctcgttccaagatct

SEQ ID NO: 4 - protein, 631 aa, Homo sapiens [sequence omitted]

SEQ ID NO: 5 - DNA, 1725 bp, Homo sapiens
  CDS (40)..(1488)
ctcatccatccgcagagcagggcagtgggaggagacgccatgacccccatcctcacggtcctgatctgtctcgggctgagtctgggccccaggacccacgtgcaggcagggcacctccccaagcccaccctctgggctgagccaggctctgtgatcatccagggaagtcctgtgaccctcaggtgtcaggggagccttcaggctgaggagtaccatctatatagggaaaacaaatcagcatcctgggttagacggatacaagagcctgggaagaatggccagttccccatcccatccatcacctgggaacacgcagggcggtatcactgtcagtactacagccacaatcactcatcagagtacagtgaccccctggagctggtggtgacaggagcctacagcaaacccaccctctcagctctgcccagccctgtggtgaccttaggagggaacgtgaccctccagtgtgtctcacaggtggcatttgacggcttcattctgtgtaaggaaggagaagatgaacacccacaacgcctgaactcccattcccatgcccgtgggtggtcctgggccatcttctccgtgggccccgtgagcccgagtcgcaggtggtcgtacaggtgctatgcttatgactcgaactctccctatgtgtggtctctacccagtgatctcctggagctcctggtcccaggtgtttctaagaagccatcactctcagtgcagccaggtcctatggtggcccccggggagagcctgaccctccagtgtgtctctgatgtcggctacgacagatttgttctgtataaggagggagaacgtgacttcctccagcgccctggttggcagccccaggctgggctctcccaggccaacttcaccctgggccctgtgagcccctcccacgggggccagtacagatgctacagtgcacacaacctctcctccgagtggtcggcccccagtgaccccctggacatcctgatcacaggacagttctatgacagaccctctctctcggtgcagccggtccccacagtagccccaggaaagaacgtgaccctgctgtgtcagtcacgggggcagttccacactttccttctgaccaaggagggggcaggccatcccccactgcatctgagatcagagcaccaagctcagcagaaccaggctgaattccgcatgggtcctgtgacctcagcccacgtggggacctacagatgctacagctcactcagctccaacccctacctgctgtctctccccagtgaccccctggagctcgtggtctcagaagcagctgagaccctcagcccatcacaaaacaagacagactccacgactacatccctaggccaacacccccaggattacacagtggagaatctcatccgcatgggtgtggctggcttggtcctggtggtcctcgggattctgctatttgaggctcagcacagccagagaagcctacaagatgcagccgggaggtgaacagcagagaggacaatgcatccttcagcgtggtggagcctcagggacagatctgatgatcccaggaggctctggaggacaatctaggacctacattatctggactgtatgctggtcatttctagagacagcaatcaatatttgagtgtaaggaaactgtctggggtgattcctagaagatcattaaactgtggtacatttttttgtctaaaaagcaggtcgtctcgttccaag

SEQ ID NO: 6 - protein, 483 aa, Homo sapiens
MTPILTVLICLGLSLGPRTHVQAGHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSEAAETLSPSQNKTDSTTTSLGQHPQDYTVENLIRMGVAGLVLVVLGILLFEAQHSQRSLQDAAGR